(19) United States
(12) Patent Application Publication
(10) Pub. No.: US 2025/0270160 A1
(43) Pub. Date: Aug. 28, 2025

(54) Processes for the Production of Vanillin and Related Compounds

(71) Applicant: UNIVERZA V LJUBLJANI, Ljubljana (SI)

(72) Inventors: Jernej Iskra, Ljubljana (SI); Monika Horvat, Ljubljana (SI)

(21) Appl. No.: 18/266,841

(22) PCT Filed: Dec. 15, 2021
(86) PCT No.: PCT/EP2021/085919

§ 371 Date: Jun. 13, 2023

(30) Foreign Application Priority Data
Dec. 16, 2020 (LU) LU102332

(51) Int. Cl. C07C 45/41

(52) U.S. Cl. CPC C07C 45/41

(57) ABSTRACT

The present invention generally relates to the production of vanillin and related compounds (such as aromatic carboxylic acids, quinones, etc.) involving the oxidative cleavage of aromatic compounds (such as lignin, lignols, cinnamic acid and its derivatives, etc.) under mild reaction conditions. More specifically, the present invention provides processes for the production of vanillin involving the oxidative cleavage of an aromatic compound, such as lignin or ferulic acid, using a peroxide based oxidant, such as hydrogen peroxide, in the presence of a suitable catalysis, such as vanadium oxide.

### TECHNICAL FIELD OF THE INVENTION

The present invention generally relates to the production of vanillin and related compounds (such as aromatic carboxylic acids, quinones, etc.) involving the oxidative cleavage of aromatic compounds (such as lignin, lignols, cinnamic acid, cinnamic acid derivatives, etc.) under mild reaction conditions. More specifically, the present invention provides processes for the production of vanillin involving the oxidative cleavage of an aromatic compound, such as lignin or ferulic acid, using a peroxide based oxidant, such as hydrogen peroxide, in the presence of a suitable catalysis, such as vanadium (V) oxide.

### BACKGROUND OF THE INVENTION

Vanillin and cinnamaldehyde are examples of naturally occurring aldehydes. Vanillin is a commonly used aromatic compound for flavoring food, pharmaceuticals and cosmetics, and it is also used as a fragrance, as a food preservative (1). About 20 000 tons of vanillin are produced annually (2). Vanillin has great potential for its use in many industries.

In recent years there has been a growing interest in the use of materials and useful chemicals from natural and waste sources. When extracting compounds from nature, it must be considered that their use does not have negative effects on the ecosystem and that no materials are used that are useful as a source of food. At the same time, it must be considered that the materials used are a continuous source of plant biomass in its natural form. The cell wall of plant biomass consists of several polymers, such as cellulose, hemicellulose, lignin, but also of minor compounds such as secondary metabolites. Cellulose is used to produce paper. During paper production a lot of waste is produced in the form of black liquor. The black liquor is an aqueous solution of lignin residues (3). Lignin is the only renewable natural aromatic biopolymer in nature (4).

Chemically, lignin consists mainly of three different units: coniferyl alcohol (G), sinapyl alcohol(S) and p-coumaryl alcohol (H). The bonds between the monolignols consist of C—C and C—O bonds (4). The structure of lignin depends on the biomass from which the lignin is obtained and the method of delignification (5). Due to its complex structure, its potential is still unexplored (6). According to data from 2016, 50 million tonnes of lignin are currently produced annually and less than 2% is used for industrial purposes (7). Lignin is thus a potential source of high value-added compounds, such as vanillin, vanillic acid, phenol etc.

Lignin can be hydrolyzed to smaller compounds by cleavage of C—C and C—O bonds, but its conversion to monomeric units is complex due to its complex structure and the mechanism of the process is therefore not yet fully understood. The synthesis of useful chemicals that can be obtained from lignin is being studied on model substrates due to its complex structure. This greatly simplifies the analysis of new products and the optimization of reaction conditions (4). The conversion of ferulic acid and other lignin model compounds into the corresponding aldehydes is achieved by oxidative cleavage of the C═C double bond.

Ferulic acid is found in the lignocellulosic biomass in plants cell walls, grasses, grains, vegetables, flowers, fruits, leaves, seeds, nuts . . . (7-10). Several methods are known for the conversion of ferulic acid into vanillin, using different microorganisms (11-16), metal oxide catalysts, TiO₂, WO₃-loaded TiO₂ (9, 17) using a photocatalytic dialysis reactor (18). The oxidation of ferulic acid has already been studied, including H₂O₂ in the presence of microorganisms. However, studies on the oxidative degradation of the carbon-carbon double bond in ferulic acid using the green oxidant hydrogen peroxide in the presence of catalysts are still unexplored. Hydrogen peroxide is an attractive oxidant, inexpensive, soluble in water and many organic solvents, and environmentally friendly, since water is the only theoretical by-product (19-21).

Overall, there remains a constant need for producing vanillin, especially for a green and environmentally friendly process for producing this compound.

### SUMMARY OF THE INVENTION

The present invention addresses this need by providing processes for the production of vanillin and related compounds (such as aromatic carboxylic acids, quinones, etc.) involving the oxidative cleavage of an aromatic compound (such as lignin, lignol or cinnamic acid and its derivatives), using a peroxide as oxidant, such as hydrogen peroxide, in the presence of a suitable catalysis, such as vanadium (V) oxide.

The advantage of the processes of the present invention is a) selectivity, as only vanillin and no other aromatic aldehydes derived from lignin are formed; b) the use of a green oxidant, such as hydrogen peroxide, which produces only water as a by-product, c) a simple and inexpensive catalyst, and d) versatility of production of other products (such as vanillic acid, quinone etc.).

The present invention thus provides a process for the production of vanillin comprising the step of reacting an aromatic compound with a peroxide in the presence of a suitable catalyst.

The present invention may be further characterized by the following items:
1. A process for the production of vanillin or a related compound comprising the step of reacting an aromatic compound with a peroxide in the presence of a suitable catalyst.
2. The process according to item 1, wherein the aromatic compound is selected from the group consisting of lignin and monomeric analogues thereof, lignols (such as coumaryl alcohol, coniferyl alcohol or sinapyl alcohol), cinnamic acid and derivatives thereof (such as ferulic acid).
3. The process according to item 1, wherein the aromatic compound is lignin or a monomeric analogue thereof.
4. The process according to item 1, wherein the aromatic compound is cinnamic acid or a derivative thereof.
5. The process according to item 1 or 4, wherein the aromatic compound is a cinnamic acid derivative according to general formula (I)

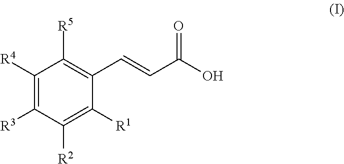

wherein R¹, R², R³, R⁴ and R⁵ are each independently selected from the group consisting is selected from the group consisting of hydrogen, hydroxyl, halogen, NO₂, C₁₋₆ alkyl optionally substituted with one or more halogens, and C₁₋₆ alkoxy optionally substituted with one or more halogens, with the proviso that at least one of R¹, R², R³, R⁴ and R⁵ is not hydrogen.
6. The process according to item 1, wherein the aromatic compound is ferulic acid.
7. The process according to any one of items 1 to 6, wherein the peroxide is selected from the group consisting of hydrogen peroxide, organic hydroperoxides, percarboxylic acids, organic peroxides and diacyloyl peroxides.
8. The process according to any one of items 1 to 7, wherein the peroxide is employed in an amount of 5 to 40 equivalents per aromatic ring of the aromatic compound.
9. The process according to any one of items 1 to 7, wherein the aromatic compound is lignin and the peroxide is employed in an amount of from 5 to 200 wt % per mass of lignin.
10. The process according to any one of items 1 to 9, wherein the peroxide is hydrogen peroxide.
11. The process according to item 10, wherein the hydrogen peroxide is employed as an aqueous solution in the range of 10% to 60%, such as an aqueous solution in the range of 25% to 35%, such as a 30% aqueous solution.
12. The process according to any one of items 1 to 11, wherein the catalyst is selected from the group consisting vanadium compounds, copper compounds, cobalt compounds, nickel compounds, ruthenium compounds, ammonium compounds and sodium compounds.
13. The process according to any one of items 1 to 12, wherein the catalyst is selected from the group consisting of a Vanadium (II) oxide (vanadium monoxide, VO), Vanadium (III) oxide (vanadium trioxide, V₂O₃), Vanadium (IV) oxide (vanadium dioxide, VO₂), Vanadium (V) oxide (vanadium pentoxide, V₂O₅), VO(acac)₂, VCl₂, VOSO₄, VOCl₃, vanadates including VO₃⁻ and VO₄³⁻ (such as Na₃VO₄ or NH₄VO₃), copper (II) bromide, ammonium metavanadate, copper (I) bromide, sodium molybdate, sodium orthovanadate, dicobalt octacarbonyl, nickel (II) chloride, and ruthenium (III) chloride.
14. The process according to any one of items 1 to 13, wherein the catalyst is a vanadium oxide.
15. The process according to item 14, wherein the vanadium oxide is selected from the group consisting of Vanadium (II) oxide (vanadium monoxide, VO), Vanadium (III) oxide (vanadium trioxide, V₂O₃), Vanadium (IV) oxide (vanadium dioxide, VO₂) and Vanadium (V) oxide (vanadium pentoxide, V₂O₅), (VO(acac) 2, VCl₂, VOSO₄, VOCl₃, vanadates including VO₃ and VO₄³⁻ (such as Na₃VO₄ and NH₄VO₃).
16. The process according to item 14 or 15, wherein the vanadium oxide is Vanadium (V) oxide.
17. The process according to any one of items 1 to 16, wherein the catalyst is employed in an amount of from 0.01 to 1 equivalent per aromatic ring of aromatic compound.
18. The process according to any one of items 14 to 16, wherein the vanadium oxide is employed in amount of 0.02 to 0.1, such as 0.05, equivalents per aromatic ring of the aromatic compound.
19. The process according to any one of items 1 to 16, wherein the aromatic compound is lignin and the catalyst is employed in an amount of from 1 to 100 wt % per mass of lignin.
20. The process according to any one of items 14 to 16, wherein the aromatic compound is lignin and the vanadium oxide is employed in amount of 2 to 10 wt %, such as 5 wt %, per mass of lignin.
21. The process according to any one of items 1 to 20, wherein the reaction is carried out in a suitable solvent.
22. The process according to item 21, wherein the solvent is selected from the group consisting of dimethoxyethane (DME), acetonitrile (MeCN), ethanol (EtOH), tetrahydrofuran (THF), methanol (MeOH), dichloromethane (DCM), heptane, ethyl acetate (EtOAc), diethyl ether, isopropanol, dimethyl carbonate (DMC) and 2,2,2-trifluoroethanol (TFE).
23. The process according to item 21 or 22, wherein the solvent is dimethoxyethane (DME) acetonitrile (MeCN) or 2,2,2-trifluoroethanol (TFE).
24. The process according to any one of items 1 to 23, wherein the reaction is carried out at a temperature in the range of 0° C. to reflux temperature.
25. The process according to any one of items 1 to 23, wherein the reaction is carried out at a temperature in the range of 10° C. to 30° C.
26. The process according to any one of items 1 to 23, wherein the reaction is carried out at a temperature in the range of 20° C. to 25° C.
27. The process according to any one of items 1 to 26, wherein the reaction is carried out for at least 10 min.
28. The process according to any one of items 1 to 26, wherein the reaction is carried out for at least 30 min.
29. The process according to any one of items 1 to 26, wherein the reaction is carried out for at least 60 min.
30. The process according to any one of items 1 to 26, wherein the reaction is carried out for at least 90 min.
31. The process according to any one of items 1 to 26, wherein the reaction is carried out for at least 120 min.
32. The process according to any one of items 1 to 28, wherein the reaction is carried out for a period of time in the range of 2 to 3 hours.
33. The process according to any one of items 1 to 32, further comprising recovering vanillin and/or said related compound.

### BRIEF DESCRIPTION OF THE FIGURES

FIG. 1: Schematic representation of lignin and lignols

FIG. 2: Conversion of ferulic acid to vanillin, vanillic acid and the corresponding quinone

FIG. 3: Conversion of various derivatives of cinnamic acid to corresponding derivatives of benzaldehydes and benzoic acids

FIG. 4: Conversion of various derivatives of hydroxycinnamic acid to corresponding derivatives of benzaldehydes, benzoic acids and benzoquinones.

FIG. 5: Reaction profile for vanillin formation.

FIG. 6: The effect of the amount of hydrogen peroxide on the oxidative cleavage of C═C double bond.

FIG. 7: Reaction profile for benzoic acid formation.

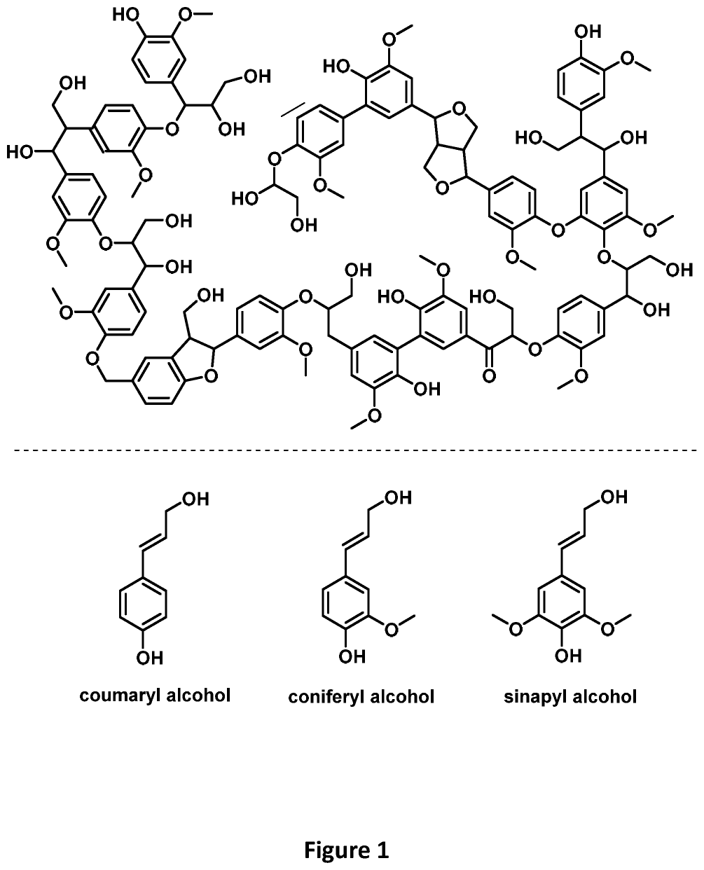

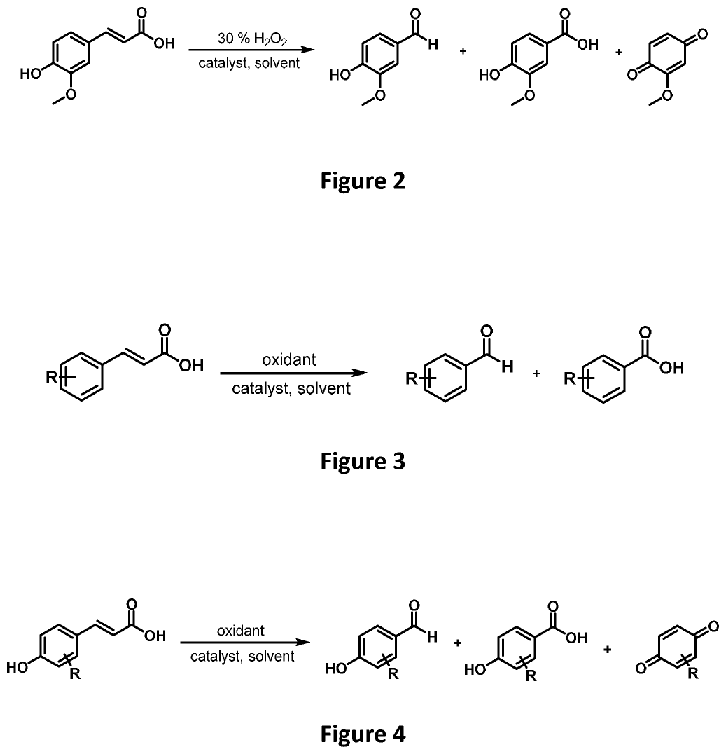

The present invention is now described in more detail below.

### DETAILED DESCRIPTION OF THE INVENTION

As noted above, the present invention is based on the surprising finding that vanillin and related compounds, such as aromatic carboxylic acids, quinones, etc., can be produced from aromatic compounds, such as lignin, lignols, or cinnamic acid and its derivatives (e.g., ferulic acid), in a green and environmentally friendly manner using a peroxide as oxidant, such as hydrogen peroxide, in the presence of a suitable catalysis, such as vanadium (V) oxide. The advantage of the processes of the present invention is a) selectivity, as only vanillin and no other aromatic aldehydes derived from lignin are formed; b) the use of a green oxidant, such as hydrogen peroxide, which produces only water as a by-product, c) a simple and inexpensive catalyst, and d) versatility of production of other products (vanillic acid, benzoic acid, benzaldehyde, quinone, etc.).

The present invention thus provides a process for the production of vanillin or a related compound comprising the step of reacting an aromatic compound with a peroxide in the presence of a suitable catalyst.

According to some embodiments, the process is for the production of vanillin.

According to some embodiments, the process is for the production of a vanillin related compound, such as vanillic acid, benzoic acid, benzaldehyde or a quinone, such as a benzoquinone. According to some embodiments, the process is for the production of vanillic acid. According to some embodiments, the process is for the production of benzoic acid. According to some embodiments, the process is for the production of benzaldehyde. According to some embodiments, the process is for the production of a quinone, such as a benzoquinone. According to some embodiments, the process is for the production of a benzoquinone, such as 2-methoxy-1,4-benzoquinone.

The aromatic compound may be any aromatic compound which comprises at least one saturated six-membered carbocyclic ring and can be converted into vanillin or a related compound. Non-limiting examples of such aromatic compounds are lignin or monomeric analogues thereof, lignols and cinnamic acids.

According to some embodiments, the aromatic compound is lignin or a monomeric analogue thereof, such as a lignol. According to some embodiments, the aromatic compound is lignin.

According to some embodiments, the aromatic compound is a lignol, such as coumaryl alcohol, coniferyl alcohol or sinapyl alcohol.

According to some embodiments, the aromatic compound is cinnamic acid or a cinnamic acid derivative. According to some embodiments, the aromatic compound is cinnamic acid. According to some embodiments, the aromatic compound is a cinnamic acid derivative.

According to some embodiments, the aromatic compound is a cinnamic acid derivative according to general formula (I)

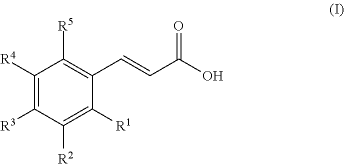

wherein R¹, R², R³, R⁴ and R⁵ are each independently selected from the group consisting is selected from the group consisting of hydrogen, hydroxyl, halogen, NO₂, C₁₋₆ alkyl optionally substituted with one or more halogens, and C₁₋₆ alkoxy optionally substituted with one or more halogens, with the proviso that at least one of R¹, R², R³, R⁴ and R⁵ is not hydrogen.

According to some embodiments, the cinnamic acid derivative is selected from the group consisting of ferulic acid, p-coumaric acid, sinapinic acid, 3-chlorocinnamic acid, 4-chlorocinnamic acid, 4-fluorocinnamic acid, 4-methylcinnamic acid, 4-bromocinnamic acid, 4-nitrocinnamic acid and 4-methylcinnamic acid.

According to some embodiments, the aromatic compound is a hydroxycinnamic acid according to general formula (II)

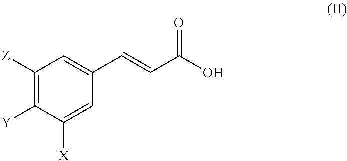

wherein X, Y and Z are each independently selected from the group consisting of hydrogen, hydroxyl, halogen, NO₂, C₁₋₆ alkyl optionally substituted with one or more halogens, and C₁₋₆ alkoxy optionally substituted with one or more halogens, with the proviso that at least one of X, Y and Z is hydroxyl.

According to some embodiments, the aromatic compound is ferulic acid, p-coumaric acid or sinapinic acid.

According to some embodiments, the aromatic compound is ferulic acid.

The aromatic compound, such as lignin or ferulic acid, may be provided as pure chemical or may be provided in the form of a composition comprising any one thereof, including a mixture thereof. For example, lignin and/or ferulic acid may be provided in the form of a lignocellulosic biomass or extract thereof. Lignin may also be provided in the form of “black liquor”, which is an aqueous solution comprising lignin residues obtained as waste after paper production. The lignin may also be in the form of organosolv lignin.

A “peroxide” within the meaning of the present invention is a compound comprising the peroxide anion O₂²⁻ or a peroxy group —O—O—. Generally, a peroxide is a compound having the structure R—O—O—R. In contrast to oxide ions, the oxygen atoms in the peroxide ion have an oxidation state of −1. Examples of a peroxide include, but are not limited to, hydrogen peroxide (H₂O₂), inorganic peroxides and organic peroxides. Examples of inorganic peroxides include, but are not limited to, barium peroxide (BaO₂), sodium peroxide (Na₂O₂), sodium percarbonate, sodium perborate. Examples of organic peroxides include, but are not limited to, compounds with the linkage C—O—O—C or C—O—O—H, such as tert-butylhydroperoxide, peracetic acid, m-chloroperbenzoic acid, dibenzoyl peroxide, diacyl peroxide or cumene hydroperoxide.

According to some embodiments, the peroxide is selected from the group consisting of hydrogen peroxide hydrogen peroxide, organic hydroperoxides, percarboxylic acids, organic peroxides, and diacyloyl peroxides. According to some embodiments, the peroxide is hydrogen peroxide.

The peroxide may be employed at any suitable amount, such as at in an amount of 5 to 40 equivalents per aromatic ring of the aromatic compound. According to some embodiments, the peroxide is employed in an amount of 5 to 30 equivalents per aromatic ring of the aromatic compound. According to some embodiments, the peroxide is employed in an amount of 6 to 8 equivalents per aromatic ring of the aromatic compound. According to some embodiments, the peroxide is employed in an amount of 7 equivalents per aromatic ring of the aromatic compound. According to some embodiments, the peroxide is employed in an amount of 20 to 30 equivalents per aromatic ring of the aromatic compound. According to some embodiments, the peroxide is employed in an amount of 2 to 8 equivalents per aromatic ring of the aromatic compound.

According to some embodiments, the aromatic compound is lignin and the peroxide is employed in an amount of from 5 to 200 wt % per mass of lignin.

According to some embodiments, the peroxide is hydrogen peroxide employed as an aqueous solution in the range of 10% to 60%. According to some embodiments, the peroxide is hydrogen peroxide employed as an aqueous solution in the range of 20% to 40%. According to some embodiments, the peroxide is hydrogen peroxide employed as an aqueous solution in the range of 25% to 35%, such as a 30% aqueous solution.

According to some embodiments, the hydrogen peroxide is employed in an amount of 5 to 40 equivalents per aromatic ring of the aromatic compound. According to some embodiments, the hydrogen peroxide is employed in an amount of 5 to 30 equivalents per aromatic ring of the aromatic compound. According to some embodiments, the hydrogen peroxide is employed in an amount of 6 to 8 equivalents per aromatic ring of the aromatic compound. According to some embodiments, the hydrogen peroxide is employed in an amount of 7 equivalents per aromatic ring of the aromatic compound. According to some embodiments, the hydrogen peroxide is employed in an amount of 20 to 30 equivalents per aromatic ring of the aromatic compound. According to some embodiments, the hydrogen peroxide is employed in an amount of 2 to 8 equivalents per aromatic ring of the aromatic compound.

It is also contemplated that the peroxide employed in accordance with the present invention is produced by a peroxidase, such as glucose oxidase, by electrolysis of water, by photocatalytic generation, or by catalytic process from oxygen.

The catalyst employed in accordance with the present invention may be any chemical compound (e.g., inorganic compound), which catalyses the oxidative cleavage of an aromatic compound, such as lignin or ferulic acid, to obtain vanillin or related compound. Non-limiting examples of suitable catalysts include vanadium compounds, copper compounds, cobalt compounds, nickel compounds, ruthenium compounds, ammonium compounds and sodium compounds.

According to some embodiments, the catalyst is selected from the group consisting of vanadium (II) oxide (vanadium monoxide, VO), Vanadium (III) oxide (vanadium trioxide, V₂O₃), Vanadium (IV) oxide (vanadium dioxide, VO₂), Vanadium (V) oxide (vanadium pentoxide, V₂O₅), VO(AcAc)₂, VCl₂, VOSO₄, VOCl₃, vanadates including VO₃⁻ and VO₄³⁻ (such as Na₃VO₄ or NH₄VO₃), copper (II) bromide, ammonium metavanadate, copper (I) bromide, sodium molybdate, sodium orthovanadate, dicobalt octacarbonyl, nickel (II) chloride, and ruthenium (III) chloride.

According to some embodiments, the catalyst is a vanadium oxide, such as a vanadium oxide selected from the group consisting of Vanadium (II) oxide (vanadium monoxide, VO), Vanadium (III) oxide (vanadium trioxide, V₂O₃), Vanadium (IV) oxide (vanadium dioxide, VO₂) and Vanadium (V) oxide (vanadium pentoxide, V₂O₅), VO(acac)₂, VCl₂, VOSO₄, VOCl₃, vanadates including VO₃ and VO₄³⁻ (such as Na₃VO₄ or NH₄VO₃). According to some embodiments, the vanadium oxide is Vanadium (V) oxide.

The catalyst may be employed at any suitable amount, such as in an amount of from 0.01 to 1 equivalents per aromatic ring of the aromatic compound. According to some embodiments, the catalyst is employed in an amount of 0.02 to 0.1, such as 0.05, equivalents per aromatic ring of the aromatic compound.

According to some embodiments, the catalyst is a vanadium oxide which is employed in amount of 0.02 to 0.1, such as 0.05, equivalents per aromatic ring of the aromatic compound.

According to some embodiments, the catalyst is a vanadium (V) oxide which is employed in amount of 0.02 to 0.1, such as 0.05, equivalents per aromatic ring of the aromatic compound.

According to some embodiments, the aromatic compound is lignin and the catalyst is employed in an amount of from 1 to 100 wt % per mass of lignin. According to some embodiments, the aromatic compound is lignin and the catalyst is employed in an amount of from 5 to 100 wt % per mass of lignin. According to some embodiments, the aromatic compound is lignin and the catalyst is employed in an amount of from 20 to 50 wt % per mass of lignin.

According to some embodiments, the aromatic compound is lignin and the catalyst is a vanadium oxide employed in an amount of from 1 to 100 wt % per mass of lignin, such as in an amount of from 20 to 50 wt % per mass of lignin.

According to some embodiments, the aromatic compound is lignin and the catalyst is a vanadium (V) oxide employed in an amount of from 1 to 100 wt % per mass of lignin, such as in an amount of from 20 to 50 wt % per mass of lignin.

Suitable, the reaction is carried out in in a suitable solvent. The solvent employed in accordance with the present invention may be any suitable solvent allowing the reaction to occur. Non-limiting examples of suitable solvents include dimethoxyethane (DME), acetonitrile (MeCN), ethanol (EtOH), tetrahydrofuran (TFA), methanol (MeOH), dichloromethane (DCM), heptane, ethyl acetate (EtOAc), diethyl ether, isopropanol, dimethyl carbonate (DMC), 2,2,2-trifluoroethanol (TFE). According to some embodiments, the solvent is dimethoxyethane (DME) or acetonitrile (MeCN). According to some embodiments, the solvent is dimethoxyethane (DME). According to some embodiments, the solvent is acetonitrile (MeCN). According to some embodiments, the solvent is 2,2,2-trifluoroethanol (TFE).

The reaction is carried out at any suitable temperature allowing the formation of vanillin, such as in the range of 0° C. to reflux temperature. According to some embodiments, the reaction is carried out at a temperature in the range of 10° C. to 60° C. According to some embodiments, the reaction is carried out at a temperature in the range of 10° C. to 30° C. According to some embodiments, the reaction is carried out at a temperature in the range of 15° C. to 60° C. According to some embodiments, the reaction is carried out at a temperature in the range of 15° C. to 30° C. According to some embodiments, the reaction is carried out at a temperature in the range of 20° C. to 60° C. According to some embodiments, the reaction is carried out at a temperature in the range of 20° C. to 30° C. According to some embodiments, the reaction is carried out at a temperature in the range of 20° C. to 25° C. According to some embodiments, the reaction is carried out at room temperature. According to some embodiments, the reaction is carried out at 60° C.

The reaction is carried out for any period of time suitable for the formation of vanillin or related product. Suitably, the reaction is carried out for at least 10 min. According to some embodiments, the reaction is carried out for at least 20 min. According to some embodiments, the reaction is carried out for at least 30 min. According to some embodiments, the reaction is carried out for at least 40 min. According to some embodiments, the reaction is carried out for at least 50 min. According to some embodiments, the reaction is carried out for at least 60 min. According to some embodiments, the reaction is carried out for at least 70 min. According to some embodiments, the reaction is carried out for at least 80 min. According to some embodiments, the reaction is carried out for at least 90 min. According to some embodiments, the reaction is carried out for at least 100 min. According to some embodiments, the reaction is carried out for at least 110 min. According to some embodiments, the reaction is carried out for at least 120 min.

According to some embodiments, the reaction is carried out for a period of time in the range of 0.5 to 24 hours. According to some embodiments, the reaction is carried out for a period of time in the range of 0.5 to 1 hour. According to some embodiments, the reaction is carried out for a period of time in the range of 2 to 3 hours. According to some embodiments, the reaction is carried out for a period of time in the range of 8 to 24 hours.

According to some embodiments, the reaction is carried out for a period of time in the range of 0.5 to 1 hours at 60° C. According to some embodiments, the reaction is carried out for a period of time in the range of 1 to 24 hours at room temperature, such as at a temperature in the range of 20° C. to 25° C. According to some embodiments, the reaction is carried out for a period of time in the range of 2 to 3 hours at room temperature, such as at a temperature in the range of 20° C. to 25° C. According to some embodiments, the reaction is carried out for a period of time in the range of 0.5 to 1 hours at 60° C. in DME. According to some embodiments, the reaction is carried out for a period of time in the range of 2 to 3 hours at room temperature, such as at a temperature in the range of 20° C. to 25° C., in DME.

According to some embodiments, the reaction is carried out for a period of time in the range of 0.5 to 1 hours at 60° C. in TFE. According to some embodiments, the reaction is carried out for a period of time in the range of 2 to 3 hours at room temperature, such as at a temperature in the range of 20° C. to 25° C., in TFE.

According to some embodiments, the reaction is carried out for a period of time in the range of 8 to 24 hours at room temperature, such as at a temperature in the range of 20° C. to 25° C., in MeCN.

According to some embodiments, the reaction is carried out for a period of time in the range of 0.5 to 1 hours at 60° C. in the presence of V₂O₅ as catalyst and hydrogen peroxide as oxidant. According to some embodiments, the reaction is carried out for a period of time in the range of 1 to 24 hours at room temperature, such as at a temperature in the range of 20° C. to 25° C. in the presence of V₂O₅ as catalyst and hydrogen peroxide as oxidant. According to some embodiments, the reaction is carried out for a period of time in the range of 2 to 3 hours at room temperature, such as at a temperature in the range of 20° C. to 25° C. in the presence of V₂O₅ as catalyst and hydrogen peroxide as oxidant.

According to some embodiments, the reaction is carried out for a period of time in the range of 0.5 to 1 hours at 60° C. in DME. According to some embodiments, the reaction is carried out for a period of time in the range of 2 to 3 hours at room temperature, such as at a temperature in the range of 20° C. to 25° C., in DME in the presence of V₂O₅ as catalyst and hydrogen peroxide as oxidant.

According to some embodiments, the reaction is carried out for a period of time in the range of 0.5 to 1 hours at 60° C. in TFE in the presence of V₂O₅ as catalyst and hydrogen peroxide as oxidant. According to some embodiments, the reaction is carried out for a period of time in the range of 2 to 3 hours at room temperature, such as at a temperature in the range of 20° C. to 25° C., in TFE in the presence of V₂O₅ as catalyst and hydrogen peroxide as oxidant.

According to some embodiments, the reaction is carried out for a period of time in the range of 8 to 24 hours at room temperature, such as at a temperature in the range of 20° C. to 25° C., in MeCN in the presence of V₂O₅ as catalyst and hydrogen peroxide as oxidant.

The process may further comprise recovering vanillin and/or the related compound. Vanillin and/or the related compound may be recovered by any conventional method for isolation and/or purification chemical compounds from a reaction. Well-known purification procedures include centrifugation or filtration, precipitation, and chromatographic methods such as e.g. ion exchange chromatography, gel filtration chromatography, etc.

Having generally described this invention, a further understanding can be obtained by reference to certain specific examples, which are provided herein for purposes of illustration only, and are not intended to be limiting unless otherwise specified.

### EXAMPLES

### Example 1—Conversion of Ferulic Acid to Vanillin

Ferulic acid 1 was selected as model substrate to investigate the optimal reaction conditions. As a starting point for our research we investigated the influence of oxidation in the presence of different catalysts (Fe₂O₃, ZrBr₂, (NH₄)₂MoO₄, RuCl₃, NiCl₂, CO₂(CO)₈, Na₃VO₄, Na₂MoO₄, CuBr, NH₄VO₃, CuBr₂, VOSO₄·H₂O, VO(acac)₂, VCl₂ and V₂O₅). The reaction was carried out using 10 mol % catalyst at room temperature for 2 h. Acetonitrile was used as solvent. During the investigation we found that a further conversion of vanillin 3 to vanillic acid 4 and 2-methoxy-1,4-benzoquinone 5 takes place.

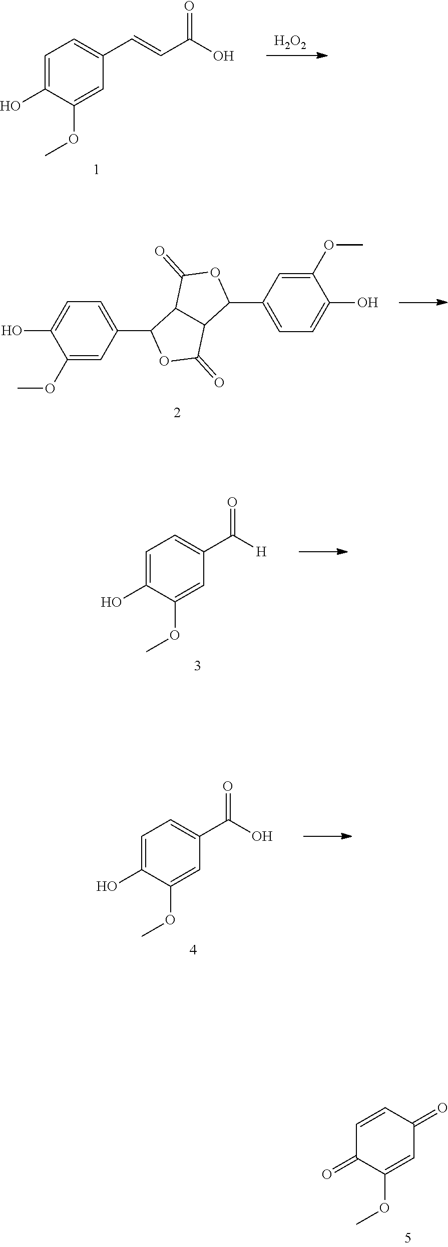

In the presence of vanadium, copper, cobalt, nickel, ruthenium, ammonium and sodium catalysts, ferulic acid 1 was converted into an intermediate product 2, vanillin 3 or one of the by-products (4, 5). The best conversion to the desired product vanillin 3 was achieved in the presence of V₂O₅.

Next, we investigated the influence of the amount of catalyst V₂O₅ on the conversion of ferulic acid 1 to vanillin 3. We tried to minimize the amount of catalyst. The results show that the amount of catalyst has no influence on the conversion. We obtained 68% of vanillin 3 when 0.01 to 1 equivalent of V₂O₅ was used. Further reactions were performed using 0.05 equivalents of catalyst V₂O₅.

TABLE 1
The oxidative cleavage of ferulic
acid with 30% aq. H₂O₂ in MeCN.

| | | | | Vanillic | |
|---|---|---|---|---|---|
| | | Vanillin | Intermediate | acid | Benzoquinone |
| entry | catalyst | 3 [%] | 2 [%] | 4 [%] | 5 [%] |
| 1 | Fe2O3 | 0 | 0 | 0 | 0 |
| 2 | ZrBr2 | 0 | 0 | 0 | 0 |
| 3 | (NH4)2MoO4 | 0 | 0 | 0 | 0 |
| 4 | RuCl3 | 0 | 2 | 0 | 0 |
| 5 | NiCl2 | 0 | 3 | 0 | 0 |
| 6 | CO2(CO)8 | 0 | 8 | 0 | 0 |
| 7 | Na2MoO4 | 0 | 17 | 0 | 0 |
| 8 | Na3VO4 | 4 | 9 | 0 | 0 |
| 9 | CuBr | 11 | 10 | 0 | 0 |
| 10 | NH4VO3 | 14 | 11 | 0 | 0 |
| 11 | CuBr2 | 39 | 21 | 8 | 0 |
| 12 | VO(acac)2 | 66 | 6 | 9 | 19 |
| 13 | VOSO4•H2O | 57 | 15 | 15 | 12 |
| 14 | VCl2 | 57 | 0 | 13 | 30 |
| 15 | V2O5 | 68 | 0 | 8 | 24 |

Reaction conditions: ferulic acid (0.1 mmol), H₂O₂ (0.7 mmol, 30%), catalyst (0.01 mmol), MeCN (1 mL), rt, 2 h.
^{a}Conversion to product was determined by 1H NMR.

Reaction conditions: ferulic acid (0.1 mmol), H₂O₂ (0.7 mmol, 30%), catalyst (0.01 mmol), MeCN (1 mL), rt, 2 h. ^{a}Conversion to product was determined by 1H NMR.

We have also tested various solvents such as acetonitrile (MeCN), ethanol (EtOH), dimethoxyethane (DME), tetrahydrofuran (THF), methanol (MeOH), dichloromethane (DCM), heptane, ethyl acetate (EtOAc), diethyl ether, isopropanol, dimethyl carbonate (DMC), dimethyl sulfoxide (DMSO), trifluoroacetic acid (TFA), water and 2,2,2-trifluoroethanol (TFE). DMSO, water and TFA are not suitable as solvents for the oxidative cleavage of the carbon-carbon double bond. The reaction was performed in the presence of TFE, DMC, isopropanol, diethyl ether, EtOAc, heptane, DCM, MeOH, THF, EtOH, MeCN or DME as solvent. The reaction was carried out quantitatively in ethanol, acetonitrile and DME. The best results were obtained in MeCN and DME, since ethyl vanillate was formed in 8% as a by-product in ethanol. In 2 h in DME as solvent the conversion to vanillin 3 was 95%.

TABLE 2
Oxidation of ferulic acid with 30% aq. H₂O₂ in different solvents.

| | | | | Vanillic | |
|---|---|---|---|---|---|
| | | Vanillin | Intermediate | aicd | Benzoquinone |
| entry | solvent | 3 [%] | 2 [%] | 4 [%] | 5 [%] |
| 1 | H₂O | 0 | 0 | 0 | 0 |
| 2 | TFA | 0 | 0 | 0 | 0 |
| 3 | DMSO | 0 | 0 | 0 | 0 |
| 4 | TFE | 0 | 0 | 0 | 100 |
| 5 | DMC | 16 | 5 | 0 | 15 |
| 6 | izopropanol | 45 | 10 | 6 | 10 |
| | diethyl | | | | |
| 7 | ether | 11 | 5 | 0 | 4 |
| 8 | EtOAc | 8 | 3 | 0 | 30 |
| 9 | heptane | 24 | 8 | 5 | 5 |
| 10 | DCM | 32 | 40 | 24 | 24 |
| 11 | MeOH | 28 | 22 | 22 | 9 |
| 12 | THF | 39 | 0 | 5 | 3 |
| 13 | EtOH | 59 | 0 | 11 | 22 |
| 14 | MeCN | 68 | 0 | 8 | 24 |
| 15 | DME | 95 | 5 | 0 | 0 |

Reaction conditions: ferulic acid (0.1 mmol), H₂O₂ (0.7 mmol, 30%), catalyst (0.005 mmol), solvent (1 mL), rt, 2 h.
^{a}Conversion to product was determined by ¹H NMR.

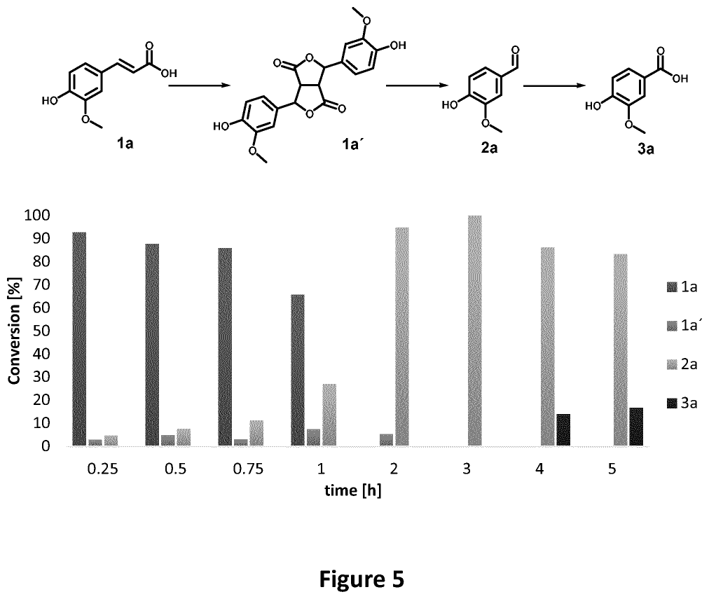

This series of experiments was carried out by varying the reaction times from 15 min to 24 h, while maintaining the amount of catalyst V₂O₅ (0.05 equiv.), the amount of 30% hydrogen peroxide solution (7 equiv.) and room temperature. The results (FIG. 5, Table 3) show the influence of the reaction time on the yield of the target product vanillin 3. When the oxidation reaction was performed out in 15 min, the yield of vanillin 3 was only 5%. In 2 h the yield increased to 100%, but an intermediate product 2 was still present in the reaction mixture. The reaction gave the best results in 3 hours. After 4 hours we got some acid (14%). A longer reaction time led to the formation of vanillic acid 4.

TABLE 3
Reaction profile for vanillin formation.

| | | | | Vanillic | |
|---|---|---|---|---|---|
| | | Vanillin | Intermediate | aicd | Benzoquinone |
| entry | solvent | 3 [%] | 2 [%] | 4 [%] | 5 [%] |
| 1 | H₂O | 0 | 0 | 0 | 0 |
| 2 | TFA | 0 | 0 | 0 | 0 |
| 3 | DMSO | 0 | 0 | 0 | 0 |
| 4 | TFE | 0 | 0 | 0 | 100 |
| 5 | DMC | 16 | 5 | 0 | 15 |
| 6 | izopropanol | 45 | 10 | 6 | 10 |
| 7 | diethyl ether | 11 | 5 | 0 | 4 |
| 8 | EtOAc | 8 | 3 | 0 | 30 |
| 9 | heptane | 24 | 8 | 5 | 5 |
| 10 | DCM | 32 | 40 | 24 | 24 |
| 11 | MeOH | 28 | 22 | 22 | 9 |
| 12 | THF | 39 | 0 | 5 | 3 |
| 13 | EtOH | 59 | 0 | 11 | 22 |
| 14 | MeCN | 68 | 0 | 8 | 24 |
| 15 | DME | 95 | 5 | 0 | 0 |

Reaction conditions: ferulic acid (0.1 mmol), H₂O₂ (0.7 mmol, 30%), catalyst (0.005 mmol), DME (1 mL), rt.
^{a}Conversion to product was determined by ¹H NMR.

The reaction was further optimized by increasing the amount of hydrogen peroxide from 2 to 10 equivalents. The reaction with 7 equivalents was able to produce vanillin 3 in 100% yield. A further increase in hydrogen peroxide resulted in a lower product formation of vanillin 3 and an increase in the amount of acid 4. We also tried different concentrations of hydrogen peroxide. The reaction also takes place in the presence of 3% hydrogen peroxide and even at a higher concentration (60%). The best results were obtained with a 30% aqueous solution of hydrogen peroxide.

TABLE 4
Oxidation of ferulic acid with different amount of H₂O₂.

| | equiv. of | Vanillin | Intermediate | vanillic acid |
|---|---|---|---|---|
| entry | H₂O₂ | 3 [%] | 2 [%] | 4 [%] |
| 1 | 2 | 22 | 8 | 0 |
| 2 | 3 | 23 | 8 | 0 |
| 3 | 5 | 54 | 9 | 0 |
| 4 | 7 | 100 | 0 | 0 |
| 5 | 7^{a} | 5 | 4 | 0 |
| 6 | 7^{b} | 10 | 4 | 0 |
| 7 | 7^{c} | 85 | 0 | 15 |
| 8 | 10 | 86 | 0 | 14 |

Reaction conditions: ferulic acid (0.1 mmol), H₂O₂ (0.2-1.0 mmol, 30%), catalyst (0.005 mmol), DME (1 mL), rt, 3 h.
^{a}Conversion to product was determined by ¹H NMR.
^{a}H₂O₂ (3%).
^{b}H₂O₂ (3%, time: 24 h).
^{c}H₂O₂ (60%).

We have also studied the influence of temperature on the implementation of the reaction. The results show that the higher temperature accelerates the conversion of the reaction. The reaction proceeds in the presence of less hydrogen peroxide and in a shorter time.

TABLE 5
Oxidation of C—C double bond at 60° C.

| | time | Equiv. of | Vanillin 3 | Intermediate 2 | vanillic acid 4 |
|---|---|---|---|---|---|
| entry | [h] | H₂O₂ | [%] | [%] | [%] |
| 1 | 2 | 2.8 | 36 | 25 | 0 |
| 2 | 2 | 4.7 | 62 | 2 | 0 |
| 3 | 1 | 7 | 100 | 0 | 0 |
| 4 | 0.5 | 7 | 100 | 0 | 0 |

Reaction conditions: ferulic acid (0.1 mmol), H₂O₂ (0.3-0.7 mmol, 30%), V₂O₅ (0.005 mmol), DME (1 mL), 60° C., time.
^{a}Conversion to product was determined by ¹H NMR.

### Preferred Procedure for Conversion of Ferulic Acid and Related Compounds to Vanillin and Corresponding Benzaldehydes

In a 10 mL volumetric flask, ferulic acid (0.5 mmol) and V₂O₅ catalyst (0.025 mmol) were added in a 5 mL solution of solvent DME. The hydrogen peroxide (30%, 7 equiv.) was first purged with argon and slowly added to a reaction mixture in three portions. The mixture was stirred at room temperature for 3 h. After completion of the reaction the reaction mixture was extracted with EtOAc (2×5 mL). The organic layer was dried over anhydrous Na₂SO₄ and evaporated under vacuum. The crude reaction was subjected to column chromatography with mobile phase EtOAc/heptane (2/1). The solvent was evaporated in vacuo to provide vanillin (91%).

### Example 2—Conversion of Ferulic Acid Model Compounds to Corresponding Benzoic Acids

Cinnamic acid 1 was taken as a model substrate, and several reaction conditions were studied to achieve oxidation to benzoic acid 4.

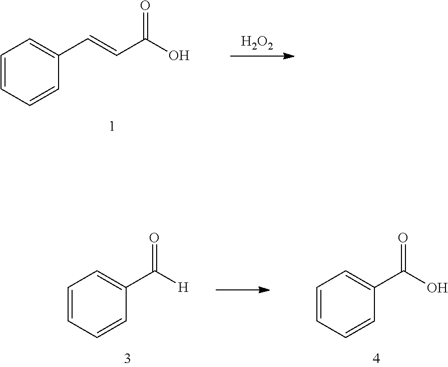 [SMILES: O=C(O)/C=C/c1ccccc1 ; "H2O2" ; O=Cc1ccccc1 ; O=C(O)c1ccccc1]

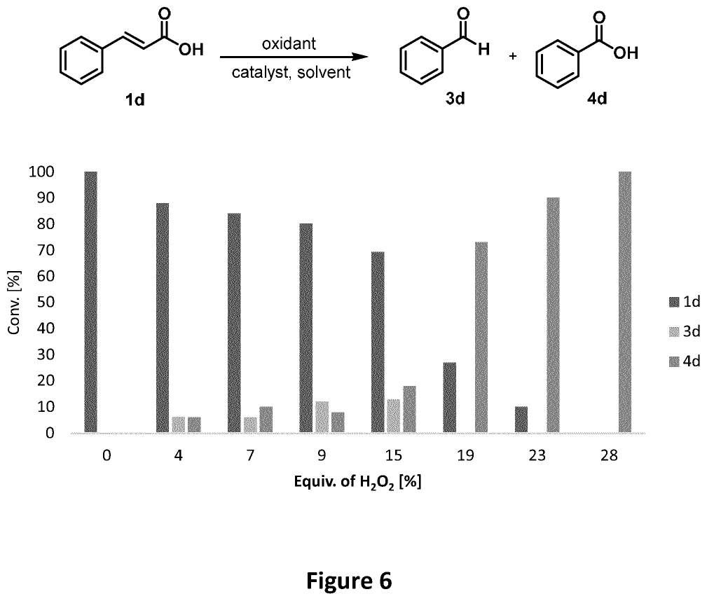

The results in FIG. 6 and Table 6 show the influence of the amount of hydrogen peroxide on the conversion of cinnamic acid 1 to benzoic acid 4. We tested the amount of hydrogen peroxide from 0 to 28 equivalents. The best results were obtained with 28 equivalents of 30% hydrogen peroxide.

TABLE 6
The effect of the amount of hydrogen peroxide
on the oxidative cleavage of C—C double bond.

| | Equiv. of | Cinnamic acid | Benzaldehyde | Benzoic acid |
|---|---|---|---|---|
| entry | H₂O₂ | 1 [%] | 3 [%] | 4 [%] |
| 1 | 0 | 100 | 0 | 0 |
| 2 | 4 | 88 | 6 | 6 |
| 3 | 7 | 84 | 6 | 10 |
| 4 | 9 | 80 | 12 | 8 |
| 5 | 15 | 69 | 13 | 18 |
| 6 | 19 | 27 | 0 | 73 |
| 7 | 23 | 10 | 0 | 90 |
| 8 | 28 | 0 | 0 | 100 |

Reaction conditions: cinnamic acid 1 (0.1 mmol), H₂O₂ (0.0-2.8 mmol, 30%), catalyst (0.005 mmol), MeCN(1 mL), rt, 24 h.
^{a}Conversion to product was determined by ¹H NMR.

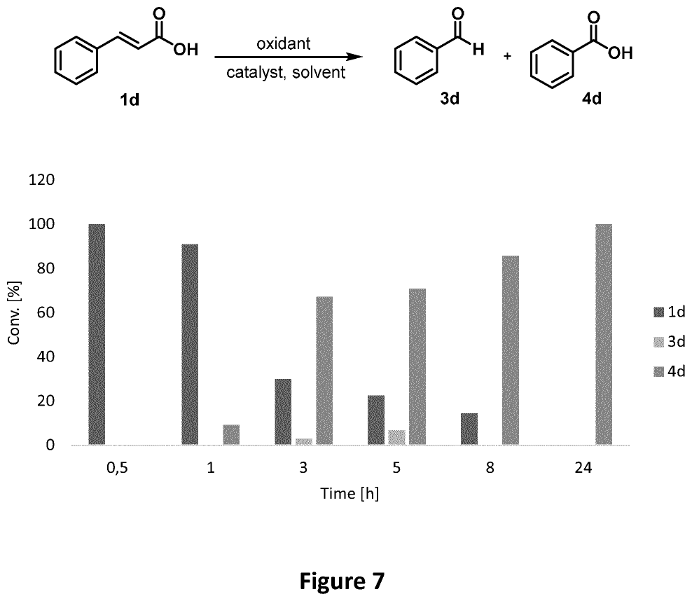

FIG. 7 and Table 7 show the effect of the reaction time on the yield of the target product benzoic acid 4. The influence of the reaction time was also tested. When the oxidation reaction was carried out in 1 h, the yield was only 9%. The reaction gave the best results in 24 hours, the cinnamic acid was completely converted into the desired product.

TABLE 7
Reaction profile for benzoic acid formation.

| | Time | Cinnamic acid 1 | Benzaldehyde 3 | Benzoic acid 4 |
|---|---|---|---|---|
| entry | [h] | [%] | [%] | [%] |
| 1 | 0.5 | 100 | 0 | 0 |
| 2 | 1 | 91 | 0 | 9 |
| 3 | 3 | 30 | 3 | 67 |
| 4 | 5 | 22 | 7 | 71 |
| 5 | 8 | 14 | 0 | 86 |
| 6 | 24 | 0 | 0 | 100 |

Reaction conditions: cinnamic acid 1 (0.1 mmol), H₂O₂ (2.8 mmol, 30%), catalyst (0.005 mmol), MeCN (1 mL), rt, time.
^{a}Conversion to product was determined by ¹H NMR.

### Preferred Procedure for Conversion Cinnamic Acid and Related Compounds to Benzoic Acid and Corresponding Aromatic Carboxylic Acids

In a 10 mL volumetric flask, substrate (0.5 mmol) and V₂O₅ catalyst (0.025 mmol) were added in a 5 mL solution of solvent MeCN. The hydrogen peroxide (30%, 28 equiv.) was first purged with argon and slowly added to a reaction mixture in three portions. The mixture was stirred for 24 h at room temperature. After completion of the reaction, the reaction mixture was extracted with EtOAc (2×5 mL). The organic layer was dried over anhydrous Na₂SO₄ and evaporated under vacuum. The crude reaction was subjected to column chromatography with mobile phase EtOAc/heptane (2/1). The solvent was evaporated in vacuo to provide the product benzoic acid (94%).

### Example 3—Conversion of Lignin to Vanillin

Lignin samples were obtained as aqueous solution after kraft pulp production of softwood materials. The conversion of lignin to vanillin was carried out according to an optimized process for the conversion of ferulic acid to vanillin. We used V₂O₅ as catalyst and DME as solvent. The reaction was carried out at room temperature. We investigated the influence of reaction time and hydrogen peroxide amount on the conversion of lignin to vanillin. The best results were achieved with 50 wt % of hydrogen peroxide in 2 h.

TABLE 8
Conversion of lignin to vanillin.

| | Time | H₂O₂ | vanillin 3 | benzoquinone 5 |
|---|---|---|---|---|
| entry | [h] | [wt %] | [%] | [%] |
| 1 | 0.25 | 50 | 0 | 0 |
| 2 | 0.5 | 50 | 7 | 1 |
| 3 | 0.75 | 50 | 10 | 2 |
| 4 | 1 | 50 | 18 | 18 |
| 5 | 2 | 50 | 83 | 17 |
| 6 | 2 | 10 | 3 | 2 |
| 7 | 2 | 75 | 71 | 29 |
| 8 | 2 | 125 | 48 | 52 |
| 9 | 1^{b} | 50 | 48 | 52 |

Reaction conditions: lignin (100 mg), H₂O₂ (10-125 wt %, 30%), catalyst (5 wt %), DME (1 mL), rt, time.
^{a}Conversion to product was determined by ¹H NMR.
^{b}solvent: MeCN.

Preferred Procedure for Conversion of Lignin to Vanillin 50 ml of black liquor of lignin were placed in Erlenmeyer flask with magnetic stirrer. Concentrated H₂SO₄ was added to lower the pH value to 2. The mixture was stirred for 10 minutes at room temperature. After completion of the precipitation process, the sample was filtered through a Büchner funnel and the filtrate was collected (lignin yield: 16 g/L). In a 5 mL volumetric flask, softwood lignin (100 mg) and V₂O₅ catalyst (5 wt %) were added in a 2 mL solution of solvent DME. The hydrogen peroxide (30%, 2×50 μL) was first purged with argon and slowly added to a reaction mixture. The mixture was stirred for 2 h at room temperature. After completion of the reaction, the reaction mixture was extracted with EtOAc (2×5 mL). The organic layer was dried over anhydrous Na₂SO₄ and evaporated under vacuum. The crude reaction was subjected to column chromatography with mobile phase EtOAc/heptane (2/1). The solvent was evaporated in vacuo to provide the product vanillin (6.1%).

### Example 4—Conversion of 4-Hydroxycinnamic Acid Derivatives to Benzoquinones

We have also developed a process for the synthesis of benzoquinones. We investigated the effect of different solvents on the conversion of ferulic acid to 2-methox-1,4-benzoquinone. The results show that the reactions occur quantitatively in TFE as solvent in the presence of V₂O₅ catalyst and hydrogen peroxide as oxidant.

TABLE 9
Oxidation of ferulic acid to 2-methoxy-1,4-benzoquinone

| | | | | 2-methoxy-1,4- |
|---|---|---|---|---|
| | | vanillin | Vanillic acid | benzoquinone |
| entry | solvent | 3 [%] | 4 [%] | 5 [%] |
| 1 | THF | 39 | 5 | 3 |
| 2 | diethyl ether | 11 | 0 | 4 |
| 3 | heptane | 24 | 5 | 5 |
| 4 | MeOH | 28 | 22 | 9 |
| 5 | izopropanol | 45 | 6 | 10 |
| 6 | DMC | 16 | 0 | 15 |
| 7 | EtOH | 59 | 11 | 22 |
| 8 | DCM | 32 | 24 | 24 |
| 9 | MeCN | 68 | 8 | 24 |
| 10 | EtOAc | 8 | 0 | 30 |
| 11 | TFE | 0 | 0 | 100 |

Reaction conditions: ferulic acid (0.1 mmol), H₂O₂ (0.7 mmol, 30%), V₂O₅ (0.005 mmol), solvent (1 mL), rt, 2 h.
^{a}Conversion to product was determined by ¹H NMR

Preferred Procedure for Synthesis of 2-Methoxy-1,4-Benzoquinone from Ferulic Acid

In a 5 mL volumetric flask, ferulic acid (0.2 mmol) and V₂O₅ catalyst (0.01 mmol) were added in a 2 mL solution of solvent TFE (2,2,2-trifluoroethanol). The hydrogen peroxide (30%, 7 equiv.) was first purged with argon and slowly added to a reaction mixture. The mixture was stirred at room temperature for 2 h. After completion of the reaction the reaction mixture was extracted with EtOAc (2×3 mL). The organic layer was dried over anhydrous Na₂SO₄ and evaporated under vacuum to provide 2-methoxy-1,4-benzoquinone (84%).

### LIST OF REFERENCES CITED IN THE DESCRIPTION

1. Ciriminna, R.; Fidalgo, A.; Meneguzzo, F.; Parrino, F.; Ilharco, L. M.; Pagliaro, M., Vanillin: The Case for Greener Production Driven by Sustainability Megatrend. ChemistryOpen 2019, 8 (6), 660-667.
2. Fache, M.; Boutevin, B.; Caillol, S., Vanillin Production from Lignin and Its Use as a Renewable Chemical. ACS Sustainable Chemistry & Engineering 2016, 4 (1), 35-46.
3. Kaur, N.; Singh, G.; Khatri, M.; Arya, S. K., Review on neoteric biorefinery systems from detritus lignocellulosic biomass: A profitable approach. Journal of Cleaner Production 2020, 256, 120607.
4. Klapiszewski, Ł.; Szalaty, T.; Jesionowski, T., Depolymerization and Activation of Lignin: Current State of Knowledge and Perspectives. 2018.
5. Sun, Z.; Fridrich, B.; de Santi, A.; Elangovan, S.; Barta, K., Bright Side of Lignin Depolymerization: Toward New Platform Chemicals. Chemical Reviews 2018, 118 (2), 614-678.
6. Guan, W.; Tsang, C.-W.; Lin, C. S. K.; Len, C.; Hu, H.; Liang, C., A review on high catalytic efficiency of solid acid catalysts for lignin valorization. Bioresource Technology 2020, 298, 122432.
7. Fache, M.; Boutevin, B.; Caillol, S., Vanillin Production from Lignin and Its Use as a Renewable Chemical. ACS Sustainable Chemistry & Engineering 2016, 4 (1), 35-46.
8. Kumar, N.; Pruthi, V., Potential applications of ferulic acid from natural sources. Biotechnology Reports 2014, 4, 86-93.
9. Gómez-López, P.; Lázaro, N.; Alvarado-Beltrán, G. C.; Pineda, A.; Balu, M. A.; Luque, R., One-Pot Cu/TiO2 Nanoparticles Synthesis for Trans-Ferulic Acid Conversion into Vanillin. Molecules 2019, 24 (21), 3985.
10. Banerjee, G.; Chattopadhyay, P., Vanillin biotechnology: the perspectives and future. Journal of the Science of Food and Agriculture 2019, 99 (2), 499-506.
11. Yang, W.; Tang, H.; Ni, J.; Wu, Q.; Hua, D.; Tao, F.; Xu, P., Characterization of Two Streptomyces Enzymes That Convert Ferulic Acid to Vanillin. PLOS ONE 2013, 8 (6), e67339.
12. Furuya, T.; Kuroiwa, M.; Kino, K., Biotechnological production of vanillin using immobilized enzymes. Journal of Biotechnology 2017, 243, 25-28.
13. Overhage, J.; Steinbüchel, A.; Priefert, H., Highly Efficient Biotransformation of Eugenol to Ferulic Acid and Further Conversion to Vanillin in Recombinant Strains of &lt;em&gt;Escherichia coli&lt;/em&gt. Applied and Environmental Microbiology 2003, 69 (11), 6569.
14. Kaur, B.; Chakraborty, D.; Kumar, B., Phenolic Biotransformations during Conversion of Ferulic Acid to Vanillin by Lactic Acid Bacteria. BioMed Research International 2013, 2013, 590359.
15. Mathew, S.; Abraham, T. E.; Sudheesh, S., Rapid conversion of ferulic acid to 4-vinyl guaiacol and vanillin metabolites by Debaryomyces hansenii. Journal of Molecular Catalysis B: Enzymatic 2007, 44 (2), 48-52.
16. Verma, A. M.; Agrawal, K.; Kawale, H. D.; Kishore, N., Quantum chemical study on gas phase decomposition of ferulic acid. Molecular Physics 2018, 116 (14), 1895-1907.
17. Parrino, F.; Augugliaro, V.; Camera-Roda, G.; Loddo, V.; Lopez-Munoz, M. J.; Marquez-Alvarez, C.; Palmisano, G.; Palmisano, L.; Puma, M. A., Visible-light-induced oxidation of trans-ferulic acid by TiO2 photocatalysis. J. Catal. 2012, 295, 254-260.
18. Camera-Roda, G.; Parrino, F.; Loddo, V.; Palmisano, L., A Dialysis Photocatalytic Reactor for the Green Production of Vanillin. Catalysts 2020, 10 (3).
19. Goti, A.; Cardona, F. In Hydrogen Peroxide in Green Oxidation Reactions: Recent Catalytic Processes, Green Chemical Reactions, Dordrecht, 2008//; Tundo, P.; Esposito, V., Eds. Springer Netherlands: Dordrecht, 2008; pp 191-212.
20. Rao, A. S.; Mohan, H. R.; Iskra, J., Hydrogen Peroxide. Encyclopedia of Reagents for Organic Synthesis, 2013.
21. Noyori, R.; Aoki, M.; Sato, K., Green oxidation with aqueous hydrogen peroxide. Chemical Communications 2003, (16), 1977-1986.

1. A process for the production of vanillin or a related compound comprising the step of reacting an aromatic compound with a peroxide in the presence of a suitable catalyst, wherein said catalyst is a vanadium compound.

2. The process according to claim 1, wherein the aromatic compound is selected from the group consisting of lignin and monomeric analogues thereof, lignols (such as coumaryl alcohol, coniferyl alcohol or sinapyl alcohol), cinnamic acid and derivatives thereof (such as ferulic acid).

3. The process according to claim 1, wherein the aromatic compound is lignin or a monomeric analogue thereof.

4. The process according to claim 1, wherein the aromatic compound is cinnamic acid or a derivative thereof or wherein the aromatic compound is ferulic acid.

5. The process according to claim 1, wherein the aromatic compound is a cinnamic acid derivative according to general formula (I) 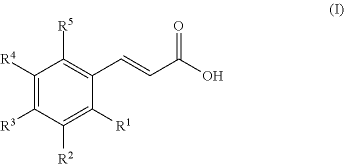
wherein R¹, R², R³, R⁴ and R⁵ are each independently selected from the group consisting is selected from the group consisting of hydrogen, hydroxyl, halogen, NO₂, C₁₋₆ alkyl optionally substituted with one or more halogens, and C₁₋₆ alkoxy optionally substituted with one or more halogens, with the proviso that at least one of R¹, R², R³, R⁴ and R⁵ is not hydrogen.

6. (canceled)

7. The process according to claim 1, wherein the peroxide is selected from the group consisting of hydrogen peroxide, organic hydroperoxides, percarboxylic acids, organic peroxides and diacyloyl peroxides.

8. The process according to claim 1, wherein the peroxide is employed in an amount of 5 to 40 equivalents per aromatic ring of the aromatic compound.

9. The process according to claim 1, wherein the aromatic compound is lignin and the peroxide is employed in an amount of from 5 to 200 wt % per mass of lignin.

10. The process according to claim 1, wherein the peroxide is hydrogen peroxide.

11. The process according to claim 10, wherein the hydrogen peroxide is employed as an aqueous solution in the range of 10% to 60%.

12. The process according to claim 1, wherein the catalyst is a vanadium oxide.

13. The process according to claim 12, wherein the vanadium oxide is selected from the group consisting of Vanadium (II) oxide (vanadium monoxide, VO), Vanadium (III) oxide (vanadium trioxide, V₂O₃), Vanadium (IV) oxide (vanadium dioxide, VO₂) and Vanadium (V) oxide (vanadium pentoxide, V₂O₅), (VO(acac) 2, VCl₂, VOSO₄, VOCl₃, vanadates including VO₃⁻ and VO₄³⁻ (such as Na₃VO₄ and NH₄VO₃).

14. (canceled)

15. The process according to claim 1, wherein the catalyst is employed in an amount of from 0.01 to 1 equivalent per aromatic ring of aromatic compound.

16. The process according to claim 12, wherein the vanadium oxide is employed in amount of 0.02 to 0.1, such as 0.05, equivalents per aromatic ring of the aromatic compound.

17. The process according to claim 1, wherein the aromatic compound is lignin and the catalyst is employed in an amount of from 1 to 100 wt % per mass of lignin.

18. The process according to claim 14, wherein the aromatic compound is lignin and the vanadium oxide is employed in amount of 2 to 10 wt %.

19. (canceled)

20. The process according to claim 1, wherein the reaction is carried out in a solvent and the solvent is selected from the group consisting of dimethoxyethane (DME), acetonitrile (MeCN), ethanol (EtOH), tetrahydrofuran (THF), methanol (MeOH), dichloromethane (DCM), heptane, ethyl acetate (EtOAc), diethyl ether, isopropanol, dimethyl carbonate (DMC) and 2,2,2-trifluoroethanol (TFE).

21. (canceled)

22. The process according to claim 1, wherein the reaction is carried out at a temperature in the range of 0° C. to reflux temperature.

23. (canceled)

24. (canceled)

25. The process according to claim 1, wherein the reaction is carried out for at least 10 min.

26. (canceled)

27. (canceled)

28. (canceled)

29. (canceled)

30. (canceled)

31. The process according to claim 1, further comprising recovering vanillin and/or said related compound.
